(19) United States
(12) Patent Application Publication
(10) Pub. No.: US 2015/0182745 A1
(43) Pub. Date: Jul. 2, 2015

(54) IONTOPHORETIC DEVICE FOR DOSAGING OF AN ACTIVE INGREDIENT

(71) Applicant: NOVAGENT OY, JORVAS (FI)

(72) Inventors: Kyösti Kontturi, Espoo (FI); Jouni Hirvonen, Helsinki (FI); Lauri Viitala, Helsinki (FI); Maija Pohjakallio, Espoo (FI)

(73) Assignee: NOVAGENT OY, JORVAS (FI)

(21) Appl. No.: 14/419,742

(22) PCT Filed: Jul. 31, 2013
(86) PCT No.: PCT/FI2013/050782

§ 371 Date: Feb. 5, 2015

(30) Foreign Application Priority Data
Aug. 13, 2012 (FI) 20120260

(51) Int. Cl. A61N 1/32; A61M 35/00

(52) U.S. Cl. CPC A61N 1/325; A61M 35/00

(57) ABSTRACT

A device based on iontophoresis and intended for transdermal dosing of an active agent, said device comprising
a pair of electrodes (11,12), which can be connected to a direct-current source, and
two chambers (13, 14), separated from each other, the first chamber (13) containing the active agent, each chamber having a porous membrane (15, 16) on the side facing the individual's skin, and
the first chamber (13) being divided in two sections (13a, 13b) in such a manner that the first chamber section (13a) is in contact with the electrode (11) and the second chamber section (13b) is in contact with the membrane (15) coming into contact with an individual's skin, wherein between the chamber sections (13a and 13b) is a membrane (18) selectively permeable either to cations or to anions,
the first chamber section (13a) containing an electrolyte and the second chamber section (13b) containing the ionic active agent, bound to a ion exchanger therein, which in turn preferably comprises fibres and ion exchanging groups bound thereto.

The invention is characterized in that
the electrodes (11,12) comprise a porous carbon fibre textile (30) which is mixed with a hydrophobic agent or onto which is fitted a hydrophobic porous, preferably micro-porous membrane (31), and
into the chamber section (13a) comprising the analyte is added a fibre grafted with buffering ion exchanging groups.

The invention relates to a device based on iontophoresis and intended for transdermal dosing of an active agent. The invention also relates to a iontophoretic device for the study of the dosing, i.e. release of an active agent.

### TECHNICAL BACKGROUND

Transdermal dosing of drugs is an established administering method for many drugs. The long-term, even and controlled concentration of a drug in the body, provided by the method, is commonly considered as an advantage of the method. By this method, side effects of the agent can be reduced and a smaller amount of a drug can be used. Also, the metabolism caused by the liver and the intestinal wall is avoided when drugs are administered transdermally.

Controlled drug release is of crucial importance in devices of this type. In the literature, release systems based on ion exchange have been presented for ionic drugs. U.S. Pat. No. 4,692,462 describes a transdermal release device wherein the drug carrier is a ion-exchange resin, which saturated with a drug and together with a salt required for the release, is mixed with a gel-form matrix. U.S. Pat. No. 6,254,883 describes a transdermal dosing system, which is based on a ion-exchange carrier in which the ion-exchange groups are grafted to a textile fiber. The publication does not, however, describe any iontophoretic device. The international published patent application WO 97/47353 describes a drug dosing assembly, based on iontophoresis, where the drug is bound to an electrically conductive carrier. The electrically conductive carrier is a textile fibre grafted with ion exchanging groups. The Finnish patent 107372 describes a iontophoresis-based drug dosing device and a device suitable for the study of the dosage. The device comprises two chamber parts, a donor chamber part, i.e. the drug containing chamber part, and an acceptor chamber part. The donor chamber is divided into two sections, an electrode section and a drug section where the sections are separated by a membrane selectively permeable to cations or to anions. The drug is bound to a ion exchanger, which for example is a textile fibre grafted with ion exchanging groups. As electrode materials are mentioned Ag/AgCl, platinum and graphite. U.S. Pat. No. 4,973,303 discloses an idea according to which the protons created at the inert electrode are buffered with a ion exchanger membrane. The ion exchanging group on the ion exchanging membrane is, depending on the sign of the electrode, —COO⁻ or —NH₃⁺. This patent does not, however, mention binding of the drug to a buffering fibre grafted with ion exchanging groups. U.S. Pat. No. 5,766,144 describes a ion exchanging system applied onto an electrode. The aim of this system is to bind the proton to the counter ion X⁻ of the ion exchanging group N⁺ at the ion exchanging polymer in which case the counter ion of the drug D⁺ moves into the polymer system. This results in the transfer of the drug across the skin according to the requirement of electro-neutrality. The drug is, however, kept in the electrolyte solution and not bound to the buffering fibre grafted with ion exchanging groups. U.S. Pat. No. 5,941,843 describes a buffering ion exchanging system applied onto an inert electrode, where the ion exchanging group is one of the following: carboxylic acid, amino, sulphonic acid or phosphoric acid group. Binding of the drug to the fibre grafted with ion exchanging groups is not mentioned. U.S. Pat. No. 7,660,626 discloses an idea to use cation exchangers to immobilize the proton and hydroxyl ion in order to raise the transport factor of the drug ion. The cell is structurally the same as that disclosed in FI 107372. The patent describes also the function of a membrane in skin contact as cation exchanger, which aims at strengthening the transport of the drug ion.

It is, however, due to numerous parameters, complicated to obtain a precise and continuously controllable skin penetration or flow of the drug.

Another remarkable problem is in that known devices are suitable for a rather short and strictly controlled use, i.e. the devices are not suitable as self-medication devices in long-term use. The electrode pair Ag/AgCl is not suitable for use in self-medication device, especially because the surface of the silver electrode in the long run becomes sticky. Another notable disadvantage related to this electrode pair is the toxicity of the electrodes and the waste problem due to this.

As electrode material can also be used an inert material such as platinum or graphite. These electrodes cause electrolysis of water so that the anode releases oxygen and hydrogen ions and the cathode releases hydrogen and hydroxyl ions. Therefore the electrodes must be porous, i.e. gas permeable. Additionally, the drug delivering space must be buffered so that the pH of the solution coming into contact with the skin remains in the physiologically suitable range.

### OBJECT AND SUMMARY OF THE INVENTION

It is an object of the present to provide an improved, transdermal device, based on iontophoresis, for dosing of an active agent, where the device does not suffer from the disadvantages of the known devices.

A particular object is to provide a device, which is suitable for long term use as self-medication device. Additionally, it shall be secured that the pH of the solution containing the active agent and coming into contact with the individual's skin remains in the physiologically suitable range. Another object is to provide an electrode the manufacture of which is easy and cheap, and which has a smooth gas permeability. Further, its surface facing the ambient air is permeable to gases but protected against moisture from the ambient air. A further object is to provide a device giving a better controllable flow than known devices.

It is also an object of the invention to provide a device based on iontophoresis for the study of dosing of active agents.

The characteristics of the present invention are given in the independent claims.

According to this invention it is essential that the electrodes comprise a carbon fibre textile and optionally a hydrophobic porous, preferably micro-porous membrane fitted onto the carbon fibre textile. The use of carbon fibre textile as electrode material for iontophoretic devices has not been suggested earlier. By use of carbon fibre textile, the manufacture of the electrode is easy and economically favourable.

Especially uniform quality of the electrode, such as smooth gas permeability, is secured.

The buffering system according to this invention, which is a fibre grafted with buffering ion exchanging groups, has several advantages over known buffering systems. Buffering salt solution require a very big space to safeguard that the device works safely for the patient over a longer time. The buffering system according to the invention is also advantageous over resins equipped with ion exchanging groups. Fibres may contain a very great amount of ion exchanging groups in proportion to the amount of fibres, and the ion exchanging groups are easily accessible. Thus, a ion to be captured (hydrogen ion or hydroxyl ion) will easily come into contact with the ion exchanging group when this is bound to a fibre, compared to a situation where the ion exchanging group is bound to a resin. The resin spheres are very big compared to the cross section of the fibre. The polymers in the resin spheres are strongly cross-linked and therefore the resin creates a steric hindrance for the motility of the ions. Thus, the solution according to this invention enables the manufacture of very compact devices which, however, work safely over a long time.

### BRIEF DESCRIPTION OF THE DRAWINGS

FIG. 1 shows a transdermal dosing device according to prior art.

FIG. 2 shows a device according to prior art, intended for the study of the release of an active agent.

FIGS. 3a and 3b show a side view of en electrode construction useful for the device according to the invention.

FIG. 4 shows a plate to be fitted in the electrode space and/or the space comprising the active agent, the plate being intended to evenly distribute the fibre material equipped with ion exchanging groups.

FIG. 5 shows the buffering effect of fibres equipped with different ion exchanging groups.

FIG. 6 shows the potential difference U as function of time during a 24 hour's iontophoretic test (in the cell: the synthetic membrane UC 010T and tacrine loaded onto Smopex®-101 ion exchanging fibre).

FIG. 7 shows the amount of tacrine in the acceptor space as function of time at the current densities 0.2 and 0.5 mA cm⁻², where the amount of tacrine per area of the membrane (UC 030T) at the current densities 0.2 mA cm⁻² (to left, four repeated tests) and 0.5 mA cm⁻² (to right, two repeated tests) is shown. The donor space contained tacrine loaded Smopex®-101 ion exchanging fibres.

FIG. 8 shows the tacrine flow during the iontophoretic run as function of current density (tacrine loaded onto Smopex®-101 ion exchanging fibres in the donor space, which was closed with a UC 010T-membrane).

FIG. 9 shows the release of tacrine from the ion exchanging fibre Smopex®-101 and Smopex®-102 in iontophoresis with a current density of 0.5 mA cm⁻². The donor space is closed with a UC 010T membrane.

FIG. 10 shows the average tacrine flows during the iontophoresis. Tacrine was loaded onto Smopex®-102 fibre in the donor space, which was closed with a UC 010T-membrane.

FIGS. 11 and 12 show the tacrine flow during the iontophoresis in cell I (FIG. 11) and in cell II (FIG. 12). In both cases tacrine is loaded onto Smopex®-102 ion exchanger fibre in the donor space, which is closed by swine epidermis.

### DETAILED DESCRIPTION OF THE INVENTION

FIG. 1 depicts a device disclosed in Finnish patent FI 107372, based on iontophoresis and intended for transdermal dosing of an active agent. The device comprises a pair of electrodes 11 and 12 which can be connected with a direct current source (not shown in the figure), as well as two chambers 13 and 14, which are separated from each other by a separating sheet 17, which in this case also serves as support for the chambers. Each chamber has a porous membrane 15 and 16, respectively, on the side facing the individual's skin 20. The first chamber 13 (donor space) is divided into two sections 13a and 13b so that the first chamber section 13a (electrode space) is in contact with the electrode 11 and the second chamber section 13b (the space for the active agent) is in contact with the membrane 15, which comes into contact with the individual's skin. Between the chamber sections 13a and 13 b is a membrane 18 selectively permeable to cations resp. anions. The first chamber space 13a comprises the electrolyte and the second section 13b comprises a ionic active agent bound to a ion exchanger therein.

The negatively or positively charged ion exchanging groups may be bound to a ion exchanging resin or to some other matrix. Preferably they are grafted to a fibre. If the drug to be dosed is cationic, negatively charged ion-exchanging groups, i.e. a cation exchanger is used. The membrane 18, which separates the chamber spaces 13a and 13b from each other is, depending on the charge sign of the active agent (the drug), a membrane selectively permeable to cations or to anions.

The membranes 15 and 16, which come into contact with the skin, are either porous membranes or porous ion exchanging membranes.

The electrolyte in the first section of the chamber 13 is preferably in solution. Alternatively, the electrolyte may be in dry form. In this case the electrolyte can be activated before the use of the device for example by adding to the space 13a an activator such as water. The electrolyte spaces are buffered with ion exchanger fibres.

The working principle of the device shown in FIG. 1 is as follows: The electrode 11 is an anode and 12 a cathode. When the electrodes are connected to a direct current source and the section 13a of the chamber 13 contains an electrolyte, the cation of the electrolyte is forced to selectively pass across the cation selective membrane 18 into the chamber section 13b, where the cationic active agent is bound to a ion exchanger. The chamber section 13b comprises also an electrolyte. When the cation moves, by means of the electrical current from the chamber section 13a to the chamber section 13b (the space comprising the active agent), from this space is transported an almost equal amount of cations through the porous, cation permeable membrane 15 and then through the skin 20. If the membrane 15 is simply a microporous membrane and not a cation selective membrane, part of the cations are lost in favour of anions. Thus, if only a microporous membrane is used, the salt concentration in the ion exchanger space, i.e. the chamber space 13b, tends to raise much stronger than if a cation selective membrane is used. A consequence of raised salt concentration in chamber section 13b is a slight decrease of the flow of active agent across the skin. The change can be notified and corrected, if needed, by adjusting the current (the effect of the direct current source is preferably adjustable). The ions transported from the device through the skin 20 into the body are Na⁺ and the cation of the active agent (the drug cation), L⁺. If the membrane 15 used is merely a microporous membrane, some Cl⁻ ions are also transported from the body through the skin. The quantity of L⁺ and Na⁺ ions transported into the body depends on the salt concentration in the ion exchanging space, i.e. the chamber section 13b, the distribution constant between the active agent and the salt, typical of the ion exchanger, and on the electrical motilities of the salt cation and the cation of the active agent. This arrangement enables the active agent to be dosed precisely, since the flow of the cations of the active agent through the skin can be determined by control of the electrical current.

The cathode chamber 14 (i.e. the acceptor space) comprises also an electrolyte.

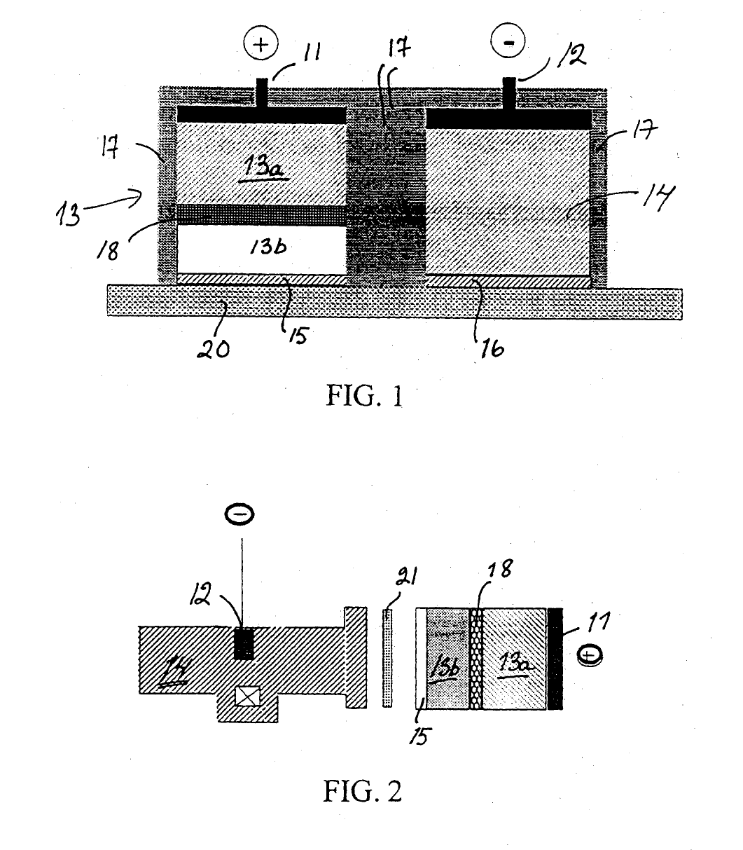

FIG. 2 shows a device disclosed in Finnish patent FI 107372, suitable for study of the release of an active agent. The device is structurally the same as the dosing device of FIG. 1, except that the individual's skin 20 is replaced with human or animal skin or with a synthetic membrane 21, and that the chamber 14 serves as a sample-taking container.

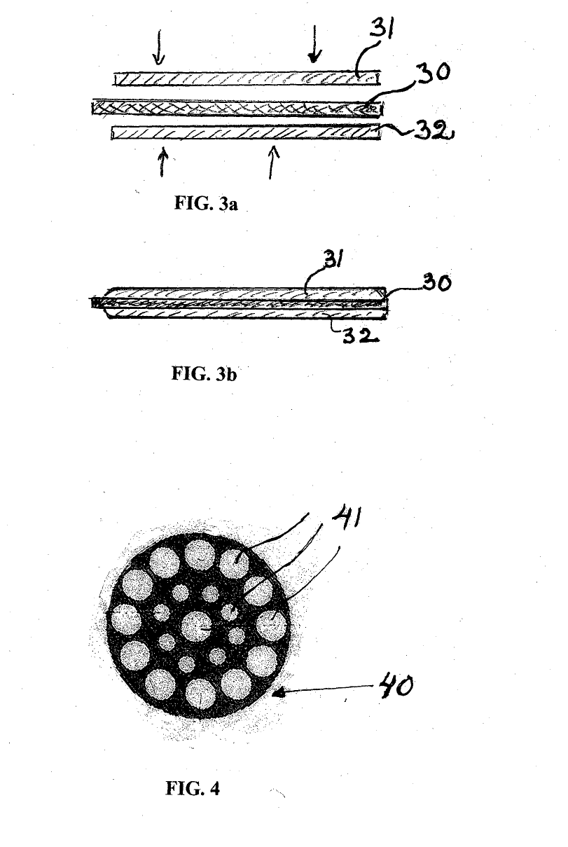

FIGS. 3a and 3b show a side view of en electrode construction useful for the device according to this invention. The electrically conductive element 30 is a carbon fibre textile. Onto the carbon fibre textile 30 is fitted a hydrophobic, porous, preferably micro-porous membrane 31, made of a polymer such as teflon. The hydrophobic membrane 31 prevents ambient moisture from entering the device. Onto the side of the carbon fibre textile 30 facing the electrolyte containing chamber section 13a is fitted a hydrophilic layer 32. This ensures that moisture comes into contact with the carbon fibre textile 30 when the device is taken into use. The layers 31, 30 and 32 shown in FIG. 3a are pressed together to an assembly shown in FIG. 3b.

Alternatively, the carbon fibre textile 30 can be admixed with a hydrophobic agent such as teflon, for example about 10%. In this case the layer 31 is not absolutely necessary.

When inert electrodes according to this invention are used, and where the electrode 11 shown in FIG. 1 is an anode and 12 a cathode, electrolysis of water takes place as follows:

$$\frac{\begin{matrix}\text{?} \text{:} 2 {H}_{2} O + 2 {e}^{-} -> {H}_{2} + 2 {OH}^{-} \\ \text{?} \text{:} {H}_{2} O -> 2 {e}^{-} + 2 {H}^{+} + \frac{1}{\text{?}} \text{?}\end{matrix}}{{H}_{2} O -> {H}_{2} + \frac{1}{\text{?}} \text{?}}$$

$$\text{?} \text{indicates text missing or illegible when filed}$$

Due to the development of gases it must thus be made sure that the gases can escape from the device. Also buffering of the electrode spaces must be carried out in order to ensure that the pH of the solution coming into contact with the individual's skin and comprising the active agent remains in a physiologically suitable range.

In the anode space hydrogen ions are released and therefore fibres grafted with buffering ion exchanging groups are added into the electrolyte containing chamber section 13a. The said ion exchanging group is an anion and works thus as a cation exchanger. In order to achieve a buffering effect, as ion exchanging group is preferably used an anion of a weak acid, such as carboxylate, i.e. —COO⁻, which binds hydrogen ions and creates the group —COOH. The carboxyl group possesses a remarkably better buffering effect than an anion of a strong acid, such as benzyl sulphonic acid anion —SO₃⁻. The test results shown below disclose a clear difference.

In the space comprising the active agent, i.e. chamber section 13b, the cationic agent is bound to the cation exchanger. The ion exchanging group of this cation exchanger is preferably also the anion of a weak acid, preferably the same anion as the ion exchanging group in chamber space 13a, i.e. most preferably carboxylate.

The electrolyte in chamber space 13a is preferably sodium sulphate, preferably an about 0.15 M aqueous solution of sodium sulphate. The use of sodium sulphate does not cause release of chlorine gas, which would be caused by use of sodium chloride. The electrolyte in chamber section 13b is, however, preferably sodium chloride, particularly a 0.15 M aqueous solution of sodium chloride, which corresponds to the physiological salt solution.

As electrolyte in the cathode chamber 14 is preferably used a solution of sodium chloride or sodium sulphate. Also into this space is added a fibre grafted with buffering ion exchanging groups. The said ion exchanging group is here a cation, preferably the cation of a weak base. Thus the ion exchanger can buffer the hydroxyl ions released by the cathode.

The membrane 18 between the chamber sections 13a and 13b, which is selectively permeable to cations, is preferably a membrane equipped with anions of a sulphonic acid. As an example can be mentioned Nafion®-115, which is a copolymer of tetrafluoroethylene and perfluorosulphonic acid.

The membrane 15, 16 facing the individual's skin 20 is preferably self-adhesive, i.e. a cation selective membrane which is self-adhesive to the skin. As a preferable material polyacrylic acid can be mentioned.

It is important that the fibre material grafted with the ion exchanging groups is evenly distributed over the cross section of the chamber sections 13a and 13b. The leveling of the compaction of the fibre material can be carried out in many ways. FIG. 4 shows a plate 40 to be fitted in the chamber spaces 13a and 13b where the plate corresponds to the cross section of the chambers and is equipped with small holes 41. Upon the plate 40, preferably on both sides of the plate 40 is bound a fibre grafted with ion exchanging groups, for example by polymerizing. It shall be stressed that the solution shown in FIG. 4 represents an example only; the leveling of the fibre material can be arranged in many other ways.

In the situation described above, the agent to be dosed is a cation. If it is desired to dose an active agent in anionic form, the electrodes shown in FIG. 1 are interchanged so that 11 is the cathode and 12 the anode. The membrane 18 shall be a membrane selectively permeable to anions. Also the membranes 15 and 16 shall be membranes selectively permeable to anions. The ion exchanger in the spaces 13a and 13b shall be an anion exchanger. In order to bind the released hydroxyl groups (i.e in order to buffer the donor space), the anion exchanger is preferably a cation of a weak base. As examples of suitable cations can be mentioned NH₄⁺, N⁺(CH₃)₃ and NH⁺(CH₃)₂.

### EXAMPLES

The invention is further described more in detail by the following examples. In the examples a device according to FIG. 2 was used. The electrodes are made of a carbon fiber textile, onto which a hydrophobic micro-porous layer of teflon is fitted. As model agent was used tacrine, which is a cationic drug. The chamber section 13a, the electrolyte space, comprised a 0.15 M aqueous solution of sodium sulphate and the chamber section 13b, the space comprising the active agent, comprised a 0.15 M aqueous solution of sodium chloride, which corresponds to a physiological salt solution. The membrane 18 between the chamber sections 13a and 13b was Nafion®-115. The membrane 15 was either a synthetic membrane (UC 101T) or swine skin. In the experiments were used Smopex®-ion exchanging fibres, made by Smoptech (Johnson Matthey). In the donor chamber part (i.e. the chamber sections 13a and 13b in FIG. 1) cation exchanging Smopex®-101- and Smopex®-102-ion exchanging fibres were investigated. The active groups of them are shown in table 1.

TABLE 1
Structure of the ion exchanging fibres used in the donor chamber part.

| Smopex ® | Functional group | |
|---|---|---|
| 101 | 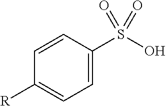 | Benzylsulponic acid |
| 102 | 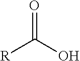 | Carboxylic acid |

Smopex®-101 contains the strong ion exchanging group SO₃⁻ and Smopex®-102 contains the weak group COO⁻. In Smopex®-101, the dry matter content of the mass was, according to the manufacturer, about 39% and in Smopex®-102 about 32%. The ion exchanging fibre aims at stabilization of the transport in the iontophoretic system, improved chemical preservability of the drug, and buffering of the electrolysis reaction at the inert electrode. The ion exchanging reactions are as follows:

Smopex®-101: SO₃⁻D⁺+y [symbol] SO₃⁻y+D⁺;

Smopex®-102: COO⁻D⁺+y [symbol] COO⁻y+D⁺;

y=Na⁺ or H⁺.

The H⁺ and OH⁻-ions created in the electrolysis reactions can thus be buffered by ion exchanging fibres. Thus, irritation of the skin, caused i.a. by pH changes when the pH deviates from the physiological window pH 3-8, can be avoided.

The buffering ability of the ion exchanging fibres is described by the buffering capacity

$$β = - \frac{d n}{d log ( {c}_{{H}^{+}} )} = \frac{d n}{d pH} , \qquad ( 1 )$$

where n is the amount of added strong base.

The equation (1) describes also the easiest way of measuring the buffering capacity. This is an acid-base titration. When moving from acidic solution towards alkaline solution by titration of the solution with a strong base, the buffering capacity is the amount of monovalent base necessary to change the pH-value by one unit.

### Example 1

### Titration Experiments

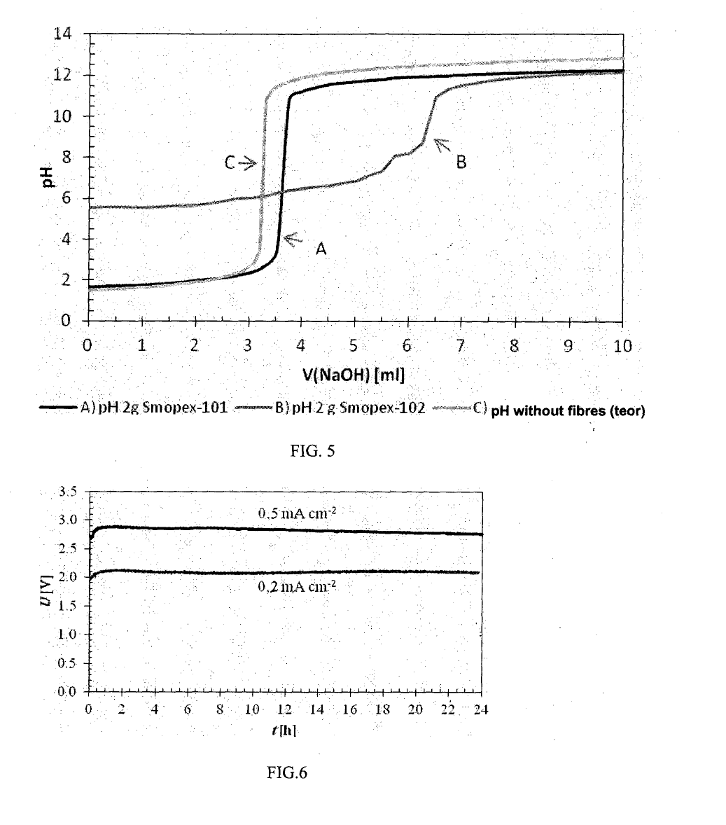

The results of the titration experiments using Smopex®-101 and Smopex®-102 are shown in FIG. 5, where the titration curves for Smopex®-101 and Smopex®-102 ion exchanging fibres are compared to a theoretical system without ion exchanging fibres.

FIG. 5 show that Smopex®-102 buffers the change of pH. On the contrary, Smopex®-101 hardly deviated from the theoretical situation without fibre. The ion exchanging fibre Smopex®-102 with weaker ion exchanging groups buffers thus the pH changes better that the stronger ion exchanging fibre Smopex®-101. The same phenomenon is also valid for anion exchangers, as for example shown by Staby et. al. J Cromatogr. A, 897 (2000), 99-111, by titration of different commercial ion exchanging resins containing different amino groups.

### Example 2

### pH-Change at the Electrodes During the Iontophoresis

The pH changes of the iontophoresis tests were compared by using different fiber systems for the drug tacrine. The pH for the electrolyte solution (0.15 M NaCl(aq)) was 6.10 without fibre. When the electrode space and the drug space comprised Smopex®-101 fibre, the pH was 6.80 before the iontophoresis. When Smopex®-102 fibre was used, the pH was 7.80. At the end of the iontophoresis run, the pH value was measured in the electrode space, the drug space and the acceptor space for about 24 h after the iontophoresis. Because H⁺-ions are released at the anode in the iontophoretic system, it is important to obtain information on how the pH in the drug space can be raised to the physiologically acceptable level (pH 3-8). The pH-values measured are shown in table 2.

TABLE 2
pH-value in the electrode space, the drug space and the acceptor space at
the end of a 24 hour's iontophoretic run. The drug space and the acceptor space were
separated by a synthetic membrane (UC 010T) or by swine skin. From repeated runs
(number of repetitions in parenthesis) the average spread was calculated.

| | Smopex ®- | 101 | | 102 | | 102 | |
|---|---|---|---|---|---|---|---|
| Current density | (membrane) | (synthetic) | | (synthetic) | | (swine skin) | |
| [mA cm⁻²] | Check point | pH | σ(n) | pH | σ(n) | pH | σ (n) |
| 0.2 | Electrode space | 2.31 | 0.05 | 3.72 | 0.36 | 4.60 | 0.36 |
| | | | (2) | | (2) | | (3) |
| | Drug space | 2.46 | | 5.31 | 0.29 | 7.13 | 0.12 |
| | | | | | (2) | | (3) |
| | Acceptor space | 9.98 | | 8.02² | 1.09² | 8.77³ | 1.33³ |
| | | | | | (2) | | (2) |
| 0.5 | Electrode space | 1.73¹ | 0.02¹ | 1.96 | | | |
| | | | (2) | | | | |
| | Drug space | 1.99¹ | 0.01¹ | 5.80 | | 4.20 | |
| | | | (2) | | | | |
| | Acceptor space | 10.90¹ | 0.15¹ | | | 7.7³ | |
| | | | (2) | | | | |

¹Smopex ® -101 was used 0.1 g resp. 0.5 g in the drug resp. electrode space. The amount had no influence on the final pH value.
²Smopex ®-102 was added in an amount of 0.2 g to the drug and electrode spaces.
The acceptor space was separated from the cathode by a salt bridge.
³When swine was used pH was buffered by using Hepes bufferts of varying strengths.

Table 2 shows that the pH remains over four in the drug space when Smopex®-102-ion exchanging fiber is used. Smopex®-101 does not buffer the pH change even at higher (0.5 g) amounts.

### Example 3

### Iontophoresis Tests Through a Neutral Membrane

The fibres Smopex®-101 and Smopex®-102 were investigated as suitable for ion exchanging fibres in the iontophoretic device. First, the Smopex®-101 fibre was investigated. Ion exchanging fibre loaded with tacrine was added to the drug space which was closed by a UC 010T ultrafiltration membrane. The iontophoresis was started and the potential was measured as function of time. A typical potential curve is shown in FIG. 6 for the current densities 0.2 mA cm⁻² and 0.5 mA cm⁻². FIG. 6 shows the potential difference U as function of time during a 24 hour's iontophoretic test (in the cell: the synthetic membrane UC 010T and tacrine loaded onto Smopex®-101 ion exchanging fibre).

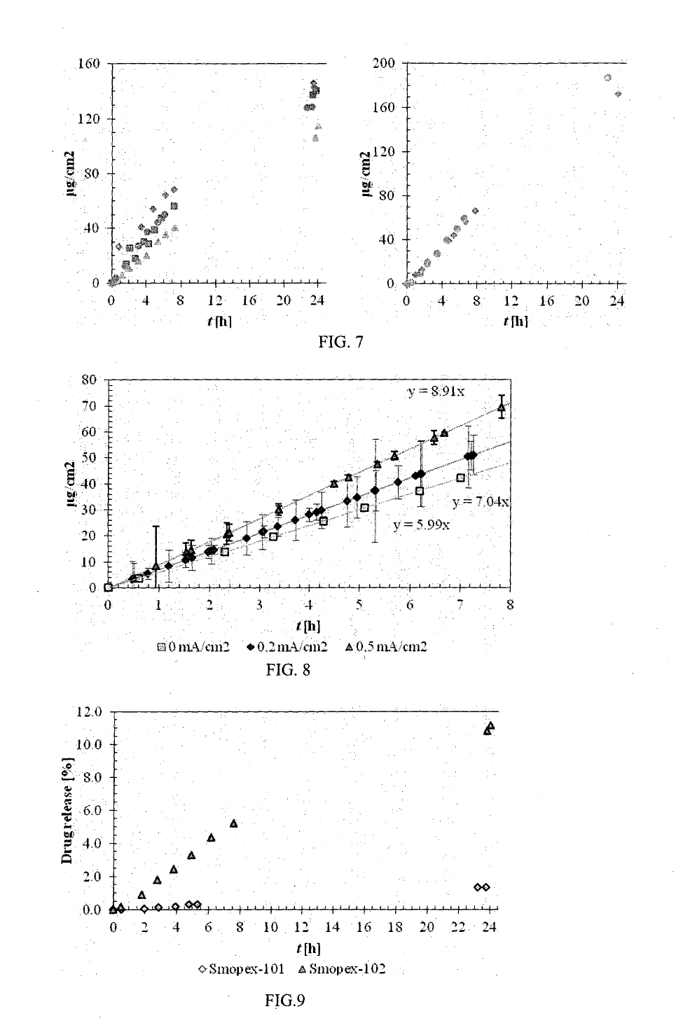

During the test a HPLC-sample was taken from the acceptor space at regular intervals for determining the tacrine flow. The amount of tacrine in the acceptor space as function of time at the current densities 0.2 and 0.5 mA cm⁻² is shown in FIG. 7, where one can see the amount of tacrine per area of the membrane (UC 030T) at the current densities 0.2 mA cm⁻² (to left, four repeated tests) and 0.5 mA cm⁻² (to right, two repeated tests). The donor space comprised tacrine loaded Smopex®-101 ion exchanging fibres.

Based on the values in FIG. 7, the average slope was determined for each current density on the linear range of the flow by forcing the start of each measurement to origo and by using the regression analysis function of the Excel program. The curves are quite linear during all the 24-hours period. The average flow and the passive diffusion (I=0) measured values for the first eight hours period are shown in FIG. 8, which shows the tacrine flow during the iontophoresis (tacrine loaded onto Smopex®-101 ion exchanger fibre in the donor space, which is closed with a UC 010T membrane).

FIG. 8 shows that increased current density increased the iontophoretic flow of tacrine. The residual of the test points are high for 0.2 mA cm⁻¹, which indicates that the error may derive from the HPLC analysis and sampling. On the other hand tacrine releases sparingly from the Smopex®-101 fibre because of the strong hydrophobic interaction between the drug tacrine and the fibre. This was also noted by calculating the tacrine content according to the values in FIG. 8 in the aqueous phase in the drug space of the cell. Additionally the transport factor and the iontophoretic strengthening constant for tacrine was determined. The results are shown in table 3.

In the second test series, the drug space of the iontophoreis cell was filled with Smopex®-102 fiber loaded with tacrine. This resulted in an essentially better tarcine flow than that obtained by the Smopex®-101 system. This is shown in FIG. 9, where the release of tacrine from the ion exchanging fibre Smopex®-101 and Smopex®-102 is shown in iontophoresis with a current density of 0.5 mA cm⁻². The donor space is closed with a UC 010T membrane.

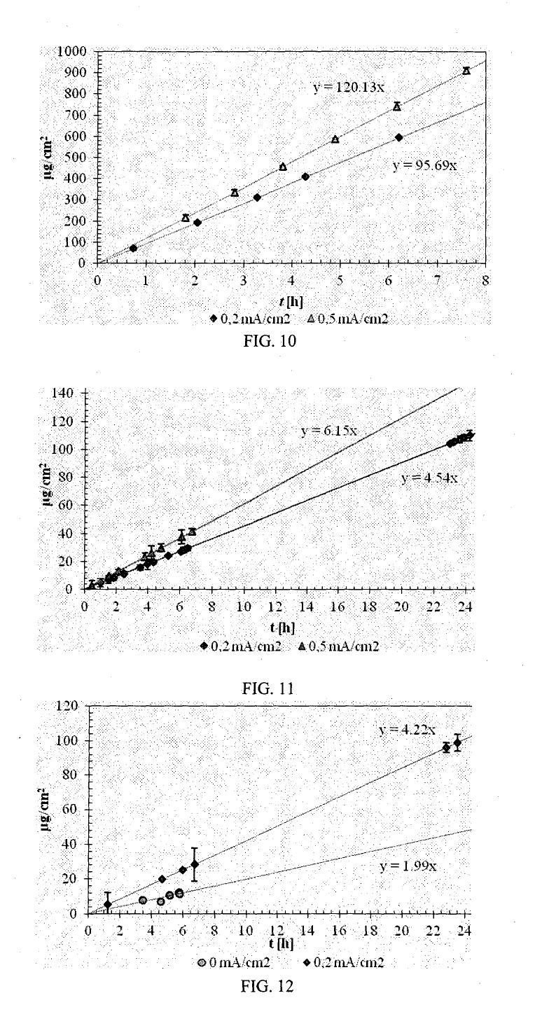

FIG. 10 shows the average tacrine flows during the iontophoresis. Tacrine was loaded onto Smopex®-102 fibre in the donor space, which was closed with a UC 010T-membrane.

Based on FIG. 9, Smopex®-102 seemed remarkably promising compared to Smopex®-101 fibre. In the Smopex®-102 system, the tacrine flow was more then tenfold. The results are of the same magnitude as those disclosed in Vuorio et. al., J Contr. rel., 97(2004), 485-92, where the tacrine values were measured in side-by-side cells (0.04 in the current density range 0.05-0.50 mA cm⁻²). However, the release of protons at the anode decreases the transport factor of tacrine (table 3).

The ratio between the flow values in FIG. 10 is 1.26. Correspondingly, the ratio for the values in FIG. 8 was 1.27. The iontophoretic flow increased thus as function of current density similarly independently of the ion exchanging fibre. Based on FIG. 10, the tacrine content in the aqueous phase in the drug space could be estimated to c_{D}≅224 μg cm⁻³, which corresponds well to the value 217 μg cm⁻³ measured by HPLC. The fiber-aqueous phase equilibrium for tacrine is more on the aqueous phase in the Smopex®-102 system than in the Smopex®-101 system (table 3).

The clearance CL of tacrine is 150 dm³h⁻¹ and the therapeutic window is 5-30 μg dm⁻³ [19.20]. Based on this fact and the flow values of both of the iontophoresis ion exchange fibre systems, it is possible to estimate the required area of the iontophoretic device for achieving the therapeutic window. The assumption of a stationary state gives the equation

$$A = \frac{CL \text{?}}{\text{?}} . \\ \text{?} \text{indicates text missing or illegible when filed} \qquad ( 2 )$$

For the membrane in contact with the skin, a required area is 106.5-639.2 cm² and 84.2-505.1 cm² for the Smopex®-101 device and 7.8-47.0 cm² and 6.2-37.5 cm² for the Smopex®-102 device at current densities 0.2 mA cm⁻² and 0.5 mA cm⁻². The diameter of a circular plaster should thus be 11.6-28.5 cm or 10.4-25.4 cm in the Smopex®-101 device and 3.2-7.7 cm or 2.8-6.9 cm in the Smopex®-102 device.

### Example 4

### Iontophoretic Tests Through Swine Skin In Vitro

The iontophorsis tests were continued by loading tacrine onto the Smopex®-102 fibre and by using swine skin instead of the synthetic membrane to close the donor space. In the test runs about 0.25 g Smopex®-102 ion exchanger fibre was loaded into the drug space. On the fibre in the drug space in prototype II, a greater amount of tacrine was loaded than in prototype I. FIGS. 11 and 12 show the test results, scaled to origo, for prototype I and II, respectively. FIG. 11 shows the tacrine flow during the iontophoresis in cell I (FIG. 11) and in cell II (FIG. 12). In both cases tacrine is loaded onto Smopex®-102 ion exchanger fibre in the donor space, which is closed by swine epidermis.

Based on the figures the transport factors for tacrine as well as the necessary contact area for obtaining a tacrine concentration achieving the therapeutic window was calculated from the flow values measured (table 3). For the passive flow in the system shown in FIG. 11 the slope 1.81 μg cm⁻²h⁻¹ was determined, wherein the concentration in the aqueous phase in the drug space was about

$$194 \frac{μg}{c \text{?}} . \\ \text{?} \text{indicates text missing or illegible when filed}$$

Correspondingly, for the system shown by FIG. 12, the passive flow was 1.99 μg cm⁻²h⁻¹ and the concentration

$$213 \frac{μg}{c \text{?}} . \\ \text{?} \text{indicates text missing or illegible when filed}$$

### Example 5

### Summary of the Iontophoresis Tests

The iontophoresis tests were started by loading the model drug, tacrine, onto the ion exchanging fibres. For two compared ion exchanging fibres (Smopex®-101 and Smopex®-102) the ion exchanging capacity X was calculated based on HPLC-analysis. In both cases, the ion exchanging fibre surpassed the ion exchanging capacity reported by the manufacturer. The reason for this is probably the fact that tacrine is lipophilic and is bound to the fibre also by the dispersion forces. The tacrine molecule is particularly lipophilic in the case of Smopex®-101 fibre because both tacrine and the ion exchanging group of the Smopex®-101 fibre contain a benzene group. The benzene groups interacts strongly with each other which results in the retention of tacrine onto the fibre. The ion exchanging capacity is generally determined by the Na⁺ and H⁺ ions, which in turn are affected only by the electrostatic interaction.

In the iontophoresis tests, Smopex®-101 did not release the drug from the ion exchanging fibre in a desired way. When the fibre was changed to Smopex®-102, the value of the iontophoretic flow density and the transport factor was improved more then tenfold (table 3). Instead, the iontophoretic intensification constant E remained on the same level as in the Smopex®-101 system, which probably is due to the lipophilicity of tacrine.

The iontophoresis tests were continued through swine epidermis using Smopex® 102 ion exchanging fibres. As result was obtained a slightly improved flow density.

TABLE 3
Results of the iontophoresis tests.

| | Smopex ®- | | Smopex ®- | | Smopex ®- | | Smopex ®-102 |
|---|---|---|---|---|---|---|---|
| Fibre | 101 | | 102 | | 102 | | (Swine |
| (membrane) | (UC 010T) | | (UC 010T) | | (Swine | | epidermis) |
| [Prototype] | I | | I | | epidermis) I | | II |
| I^{(a)} | 0.20 | 0.50 | 0.20 | 0.50 | 0.20 | 0.50 | 0.20 |
| [mA cm⁻²] | | | | | | | |
| Flow densiy | | | | | | | |
| J_{if} ^{(b)} | 7.04 | 8.91 | 95.69 | 120.13 | 4.54 | 6.15 | 4.22 |
| [μg cm⁻² h⁻¹] | | | | | | | |
| (E)^{(c)} | 1.15 | 1.59 | 1.15 | 1.59 | 2.51 | 3.40 | 2.12 |
| (Δφ)^{(d)} | 7.3 | 25.9 | 7.3 | 25.9 | 57.8 | 84.3 | 45.0 |
| [mV] | | | | | | | |
| (t)^{(e)} | 4.1 | 2.3 | 53 | 32 | 3.1 | 1.7 | 2.9 |
| 10³ | | | | | | | |
| Aₘᵢₙ^{(f)} | 106.5 | 84.2 | 7.8 | 6.2 | 165.2 | 122.0 | 177.7 |
| [cm²] | | | | | | | |
| c^{b(g)} | 17 | | 224 | | 194 | | 213 |
| [μg cm⁻³] | | | | | | | |

^{(a)}Current density
^{(b)}Flow density
^{(c)}Iontophoretic intensification constant.
^{(d)}Potential space
^{(e)}Transport factor
^{(f)}Required area of the membrane in contact with the skin for obtaining a tacrine flow reaching the minimum level of the therapeutic window
^{(g)}Tacrine level in the aqueous phase of the drug
The ion exchanging fibres enable a controlled drug dosing in a iontophoretic system. The use of ion exchanging fibres may remarkably improve also the chemical preservability of the drug, which enables storing and iontophoretic dosing also of less stable charged drugs in a more controllable way. In this study, Smopex ®-102 ion exchange fibre appeared to be a useful alternative when the tested drug was tacrine.

The embodiments of the invention disclosed above are examples only of the implementation of the inventive idea. It is apparent for the skilled person that the various embodiments will vary within the scope of the claims presented below.

1-16. (canceled)

17. An iontophoresis device for transdermal dosing of an ionic active agent to an individual, said device comprising
a first electrode (11) and a second electrode (12), each of which can be connected to a direct-current source, and
a first chamber (13) and a second chamber (14) separated from each other, the first chamber (13) containing the ionic active agent, each chamber having a porous membrane (15, 16) on a side of the device to be in contact with the individual's skin,
wherein the first chamber (13) is divided into a first section (13a) and a second section (13b) separated by a membrane (18) selectively permeable to either cations or anions, wherein the first chamber section (13a) is in contact with the first electrode (11) and the second chamber section (13b) is in contact with the porous membrane (15),
wherein the first chamber section (13a) contains an electrolyte and the second chamber section (13b) contains the ionic active agent bound to an ion exchanger, wherein the ion exchanger preferably comprises fibres and ion exchanging groups bound thereto,
wherein the first electrode (11) and second electrode (12) each comprises a permeable carbon fibre textile (30), wherein the carbon fibre textile (30) (a) is mixed with a hydrophobic agent or (b) a hydrophobic porous membrane (31), preferably a hydrophobic micro-porous membrane, is fitted onto the carbon fibre textile (30), and
wherein the first chamber section (13a) further contains a fibre grafted with buffering ion exchanging groups.

18. The device according to claim 17, wherein the hydrophobic micro-porous membrane (31) comprises a hydrophobic polymer, for example teflon.

19. The device according to claim 17, wherein the side of the carbon fibre textile (30) facing the chamber section (13a).

20. The device according to claim 17, wherein the buffering ion exchanging group in the chamber section (13a) is a cation exchanger, which is the anion of a weak acid, or an anion exchanger, which is the cation of a weak base.

21. The device according to claim 17, wherein the ion exchanger in the chamber section (13b) comprises the same ion exchanging group as that in the chamber section (13a).

22. The device according to claim 17, wherein the fiber grafted with ion exchanging groups is evenly distributed over the cross section of the chamber sections (13a and 13b).

23. The device according to claim 17, wherein each of the chamber sections (13a, 13b) compresses a plate (40), fitted therein, wherein each plate (40) corresponds to the cross section of chamber sections (134, 135), wherein each plate (40) is equipped with holes (41), and wherein a fiber grafted with ion exchanging groups is bound onto said plate.

24. The device according to claim 17, wherein the membrane (15, 16) facing the individual's skin (20) is a porous membrane or a porous ion exchanging membrane, preferably a self-adhesive membrane, permeable to cations resp. anions.

25. The device according to claim 24, wherein the self-adhesive membrane (15, 16) comprises polyacrylate.

26. The device according to claim 17, wherein the electrode (11) in the first chamber section (13a) of the first chamber (13) is an anode; the active agent is a cation; the ion exchanger is a cation exchanger, wherein the ion exchanging group preferably is the anion of a weak acid; the membrane (18) between the chamber sections (13a and 13b) is a membrane selectively permeable to cations; and that the membranes (15, 16) facing the individual's skin (20) are membranes permeable to cations.

27. The device according to claim 17, wherein the electrode (11) in the first chamber section (13a) of the first chamber (13) is a cathode; the active agent is an anion; the ion exchanger is an anion exchanger, wherein the ion exchanging group preferably is the cation of a weak base; the membrane (18) between the chamber sections (13a and 13b) is a membrane selectively permeable to anions; and that the membranes (15, 16) facing the individual's skin (20) are membranes permeable to anions.

28. An device for studying transdermal dosing of an ionic active agent, said device comprising
a first electrode (11) and a second electrode (12), each of which can be connected to a direct-current source, and
a first chamber (13) and a second chamber (14) separated from each other, the first chamber (13) containing the ionic active agent, the first chamber (13) having a porous membrane (15) on a side of the device to be in contact with skin (21), and the second chamber (14) comprising a suitable solvent,
wherein the first chamber (13) is divided into a first section (13a) and a second section (13b) separated by a membrane (18) selectively permeable to either cations or anions, wherein the first chamber section (13a) is in contact with the first electrode (11) and the second chamber section (13b) is in contact with the porous membrane (15),
wherein the first chamber section (13a) contains an electrolyte and the second chamber section (13b) contains the ionic active agent bound to an ion exchanger, wherein the ion exchanger preferably comprises fibres and ion exchanging groups bound thereto,
wherein the first electrode (11) and second electrode (12) each comprises a permeable carbon fibre textile (30), wherein the carbon fibre textile (30) (a) is mixed with a hydrophobic agent or (b) a hydrophobic porous membrane (31), preferably a hydrophobic micro-porous membrane, preferably a hydrophobic polymer, preferably teflon, is fitted onto the carbon fibre textile (30), wherein a side of the carbon fibre textile (30) facing first chamber section (13a) is fitted with a hydrophilic layer, and
wherein the first chamber section (13a) further contains a fibre grafted with buffering ion exchanging groups.

29. The device according to claim 28, wherein the buffering ion exchanging group in the chamber section (13a) is a cation exchanger, which is the anion of a weak acid, respectively an anion exchanger, which is the cation of a weak base.

30. The device according to claim 28, wherein the fiber grafted with ion exchanging groups is evenly distributed over the cross section of the chamber sections (13a and 13b), for example so that into the chamber sections (13a, 13b) has been fitted a plate (40), corresponding to their cross section and equipped with holes (41), wherein a fiber grafted with ion exchanging groups is bound onto said plate.

31. The device according to claim 28, wherein the electrode (11) in the first chamber section (13a) of the first chamber (13) is an anode; the active agent is a cation; the ion exchanger is a cation exchanger, wherein the ion exchanging group preferably is the anion of a weak acid; the membrane (18) between the chamber sections (13a and 13b) is a membrane selectively permeable to cations; and that the membrane (15) facing the piece of skin (21) is permeable to cations.

32. The device according to claim 28, wherein the electrode (11) in the first chamber section (13a) of the first chamber (13) is a cathode; the active agent is an anion; the ion exchanger is an anion exchanger, wherein the ion exchanging group preferably is the cation of a weak base; the membrane (18) between the chamber sections (13a and 13b) is a membrane selectively permeable to anions; and that the membrane (15) facing the piece of skin (21) is permeable to anions.
